(19) United States
(12) Patent Application Publication
(10) Pub. No.: US 2006/0122478 A1
(43) Pub. Date: Jun. 8, 2006

(54) Occupational safety product with contactless measuring electrodes

(75) Inventors: Robert Sliepen, Aachen (DE); Frank Sattler, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: Drager Safety AG & Co. KGaA, Lubeck (DE)

(21) Appl. No.: 11/231,116

(22) Filed: Sep. 20, 2005

(30) Foreign Application Priority Data
Dec. 7, 2004 (DE) 10 2004 058 781.7

(51) Int. Cl. A61B 5/04

(52) U.S. Cl. 600/383; 600/388; 600/393

(57) ABSTRACT

An occupational safety product and system has at least one contactless, especially capacitive measuring electrode (2) for detecting physiological parameters of the user of the occupational safety product makes possible the rapid and reliable adaptation of the electrodes at a defined distance from the skin surface. The measuring electrodes (2) are integrated for this, for example, directly in the rubber parts of the mask body of the breathing mask (1) or in the strap (3) of the mask. A reference electrode (33) and two contactless measuring electrodes (2) are connected to an evaluating unit (40) for ECG signals via electric lines (50).

### CROSS REFERENCE TO RELATED APPLICATIONS

This application claims the benefit of priority under 35 U.S.C. § 119 of German Application DE 10 2004 058 781.1 filed Dec. 7, 2004, the entire contents of which are incorporated herein by reference.

### FIELD OF THE INVENTION

The present invention pertains to an occupational safety product with at least one contactless measuring electrode

### BACKGROUND OF THE INVENTION

Electrodes commonly used hitherto are used to measure physiological parameters on the basis of time-dependent potentials for an electrocardiogram (ECG) or the heart rate (HR) of the user of occupational safety products such as breathing masks or respirators. The physical load during the performance of work is thus monitored. The integration of the physiological sensors, such as ECG electrodes used so far in the protective gear of users of respirators usually requires direct contact with the skin. For example, ECG electrodes consist of a silver-silver chloride combination and are applied with a gel-like electrolyte. The contact resistance varies, among other things, with the pressure with which the electrodes are applied and the properties of the skin surface of the user. In addition, there exist electrochemical potentials called half-cell potentials at the transition between electrodes and electrolyte, which lead to d.c. signals and make signal processing difficult because of the offset voltages that are variant in time.

ECG measuring set-ups, in particular, require a reference electrode on the body which must be arranged additionally, so that a direct galvanic connection is established with the body at a suitable site. The rapid and reliable arrangement of measuring electrodes with measuring cables has not been easy to bring about so far and is not feasible in every region of the body, because integration in occupational safety products such as breathing masks or respirators, such as compressed air breathing apparatus, is possible with difficulty at best. The reliability and the durability of the application of the electrodes is additionally greatly compromised by the work typically performed by users of occupational safety products.

### SUMMARY OF THE INVENTION

The object of the present invention is to improve the measuring electrodes in terms of handling and measurement for users of occupational safety products, so that the adaptation of the measuring electrodes is possible in a short time and reliably at a defined distance from the skin surface. In addition, a reference electrode can be integrated in the occupational safety product.

According to the invention an occupational safety product is provided with at least one said contactless measuring electrode for detecting physiological parameters of the user of the occupational safety product.

Due to the use of contactless measuring electrodes and especially due to the use of measuring electrodes detecting the voltage potentials capacitively, as is described, for example, in U.S. Pat. No. 6,686,800 B2, a direct galvanic skin contact of the measuring electrodes is no longer necessary. Measuring electrodes detecting voltage potentials capacitively also operate through one or more layers of textile, so that they can also be applied for applications other than users of protective clothes. The most constant distance possible between the skin of the body/body surface and the measuring electrode, which is determined by the geometry of the occupational safety product used for the measuring electrodes, should be ensured.

The occupational safety product may be a breathing mask and the at least one contactless measuring electrode may be incorporated in the mask body or in the strap of the mask.

The occupational safety product may be a respirator and the at least one contactless measuring electrode may be incorporated in the respirator itself, in the carrying device of the respirator or in the carrying belt of the respirator.

The occupational safety product may be protective clothes of the user and the at least one contactless measuring electrode may be incorporated in the protective clothes.

The at least one contactless measuring electrode may be an electrode capacitively detecting the electric voltage potential of the user of the respirator product.

The at least one measuring electrode may be used, together with the additional electrodes, to record an electrocardiogram and/or to detect the heart rate of the user of the occupational safety product.

A reference electrode may be present in the occupational safety product that is in direct contact with the skin of the user of the occupational safety product.

The at least one measuring electrode and/or the reference electrode may be connected to a evaluating unit for the measured signals of the electrodes, which is worn by the user of the occupational safety product. The evaluating unit may be connected to a mission command center for data transfer in a wireless manner.

The contactless measuring electrodes used can be installed in a breathing mask, in a respirator, such as a compressed air breathing apparatus, or the protective clothes of, for example, a firefighter. The drawbacks described in the introduction are avoided now and no additional handling steps are necessary to place and fix the measuring electrodes for detecting physiological parameters correctly at a defined distance from the skin surface on the user's body.

In particular, slipping of the measuring electrodes is also avoided during the mission of the user of the occupational safety product. The reference electrode necessary for an ECG measurement can likewise be arranged at the occupational safety product, but in direct contact with the user's skin.

Exemplary embodiments of the present invention will be described below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an occupational safety product in the form of a breathing mask and a piece of clothing;

FIG. 2 is a schematic view showing an occupational safety product in the form of a respirator with a carrying device and carrying belt belonging to it; and

FIG. 3 is a schematic view showing an occupational safety product in the form of protective clothes.

### DESCRIPTION OF THE PREFERRED EMBODIMENTS

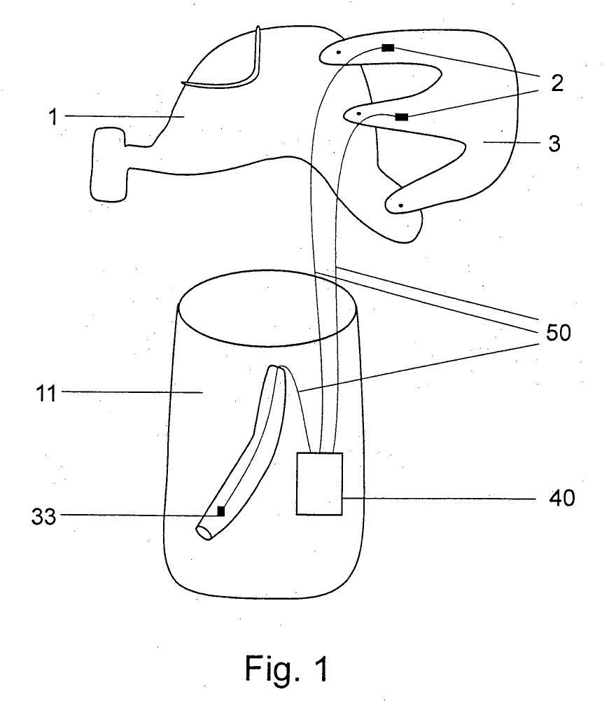

Referring to the drawings in particular, FIG. 1 shows possible placements for one or more contactless measuring electrodes 2 on a breathing mask 1. The measuring electrodes 2, whose number equals two in this case, are preferably incorporated directly in the rubber parts of the mask body and are thus also protected from environmental effects. As an alternative, the measuring electrodes 2 are attached or clipped on, fixed to the mask body, In another embodiment, the measuring electrodes 2 can also be incorporated especially in the strap 3 of the mask. A reference electrode 33 is incorporated in the piece of clothing 11, which is likewise shown schematically, in the area of the wrist for direct skin contact with the user of the piece of clothing 11. Such a reference electrode 33 is necessary for recording an ECG. The great distance between the reference electrode 33 and the measuring electrodes 2 improves the quality of the ECG measurement. The electrodes are connected to an evaluating unit 40 for the measured signals of the electrodes, which is carried, for example, on the body of the user of the occupational safety product, by means of electric lines 5. The evaluating unit 40 may be connected with a mission command center in a wireless manner for data transfer.

If it is ensured by the dimensioning of the occupational safety product at the site of the measuring electrodes 2 that the distance between the measuring electrodes 2 and the user's body is only a few mm, a measured signal quality that corresponds at least to [that of] the conventional electrodes with direct skin contact is obtained.

The ECG signals determined from the measured signals in the evaluating unit 40 are optionally stored or can be sent, for example, to a mission command center of the fire station by means of a line or even in a wireless manner.

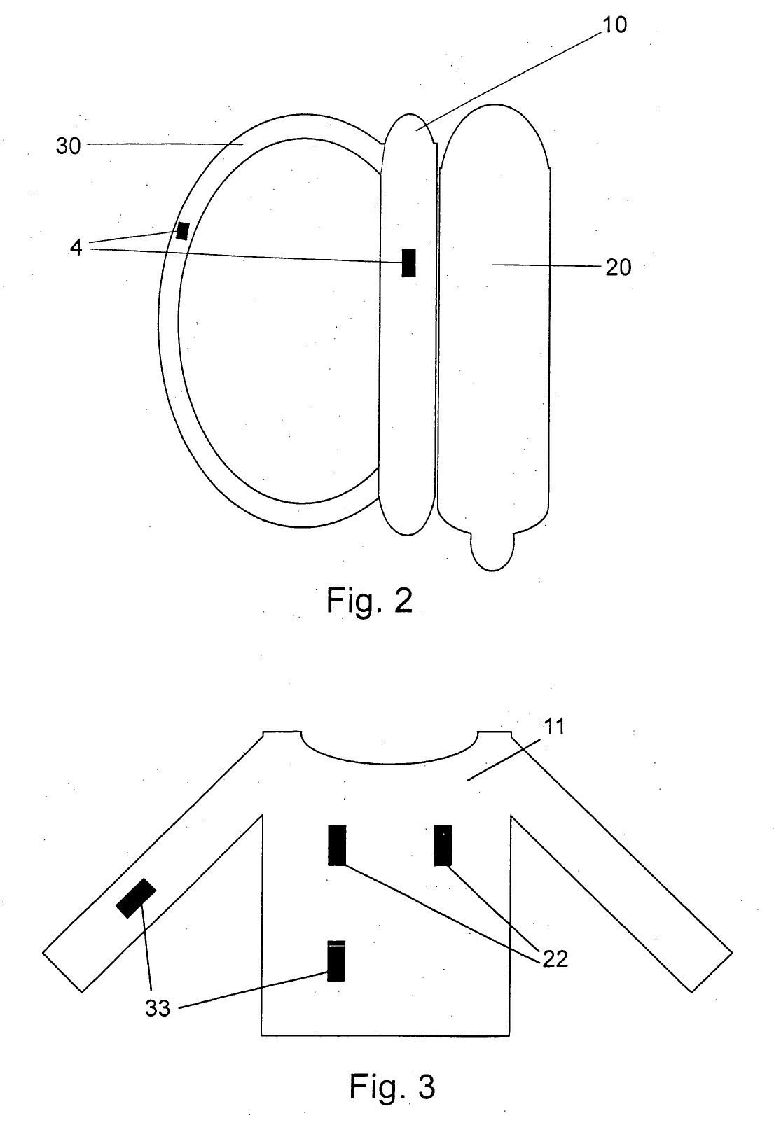

FIG. 2 shows possible sensor positions 4 for measuring electrodes 2 in the carrying belt 30 or in the carrying device 10 for the compressed air cylinder 20 of a respirator.

FIG. 3 shows protective clothes 11, for example, underwear or a jacket with possible sensor positions 22 for measuring electrodes 2. Possible positions for a reference electrode 33 must be located at a sufficiently great distance from the sensor positions 22 for the measuring electrodes 2. In this case of the devices according to FIGS. 1 through 3, the distance between the reference electrode positions and the measuring electrodes 2 is substantially greater than the distance between the measuring electrodes 2.

While specific embodiments of the invention have been shown and described in detail to illustrate the application of the principles of the invention, it will be understood that the invention may be embodied otherwise without departing from such principles.

What is claimed is:

1. An occupational safety product, comprising:
an occupational safety product part; and
at one said contactless measuring electrode for detecting physiological parameters of the user of the occupational safety product, said at one said contactless measuring electrode being connected to said occupational safety product part.

2. An occupational safety product in accordance with claim 1, wherein the occupational safety product is a breathing mask and the an occupational safety product part is a mask body or the strap of the mask and said at least one contactless measuring electrode is incorporated in the mask body or in the strap of the mask.

3. An occupational safety product in accordance with claim 1, wherein said occupational safety product is a respirator and the an occupational safety product part is a respirator part, the carrying device of the respirator or a carrying belt of the respirator and said at least one contactless measuring electrode is incorporated in the respirator part, in the carrying device of the respirator or in the carrying belt of the respirator.

4. An occupational safety product in accordance with claim 1, wherein said occupational safety product is protective clothes of the user and said at least one contactless measuring electrode is incorporated in the protective clothes.

5. An occupational safety product in accordance with claim 1, wherein said at least one contactless measuring electrode is an electrode capacitively detecting the electric voltage potential of the user of the respirator product.

6. An occupational safety product in accordance with claim 1, further comprising an additional electrode wherein said at least one measuring electrode is used, together with said additional electrode, to record an electrocardiogram and/or to detect the heart rate of the user of the occupational safety product.

7. An occupational safety product in accordance with claim 1, further comprising a reference electrode associated with the occupational safety product in direct contact with the skin of the user of the occupational safety product.

8. An occupational safety product in accordance with claim 7, further comprising an evaluating unit wherein said at least one measuring electrode and/or said reference electrode is connected to said evaluating unit for the measured signals of the electrodes, said evaluating unit being worn by the user of the occupational safety product.

9. An occupational safety product in accordance with claim 8, wherein said evaluating unit is connected to a mission command center for data transfer in a wireless manner.

10. An occupational safety product system, comprising:
an occupational safety product part worn by a user of the occupational safety product; and
a contactless measuring electrode supported by said occupational safety product part for contactless measurement of physiological parameters of the user of the occupational safety product.

11. A safety product system in accordance with claim 10, wherein the occupational safety product part comprises one of a breathing mask body or a mask strap and said contactless measuring electrode is incorporated in material forming said mask body or said strap.

12. An occupational safety product in accordance with claim 10, wherein said occupational safety product part is a respirator part, a carrying device of the respirator or a carrying belt of the respirator and said contactless measuring electrode is incorporated in the material forming the respirator part, in the carrying device of the respirator or in the carrying belt of the respirator.

13. A safety product system in accordance with claim 10, wherein said occupational safety product part is an article of protective clothes and said contactless measuring electrode is incorporated in said article of protective clothes.

14. A safety product system in accordance with claim 10, wherein said contactless measuring electrode is an electrode capacitively detecting the electric voltage potential of the user of the respirator product.

15. A safety product system in accordance with claim 10, further comprising an additional electrode wherein said at least one measuring electrode is used, together with said additional electrode, to record an electrocardiogram and/or to detect the heart rate of the user of the occupational safety product.

16. A safety product system in accordance with claim 15, further comprising a reference electrode associated with the occupational safety product in direct contact with the skin of the user of the occupational safety product.

17. A safety product system in accordance with claim 16, further comprising an evaluating unit wherein said at least one measuring electrode and/or said reference electrode is connected to said evaluating unit for the measured signals of the electrodes, said evaluating unit being worn by the user of the occupational safety product.

18. A safety product system in accordance with claim 17, wherein said evaluating unit is connected to a mission command center for data transfer in a wireless manner.
